(19) United States
(12) Patent Application Publication
(10) Pub. No.: US 2025/0144428 A1
(43) Pub. Date: May 8, 2025

(54) MEDICAL ARRANGEMENT FOR ELECTRICAL NEUROSTIMULATION

(71) Applicant: B. Braun Melsungen AG, Melsungen (DE)

(72) Inventor: Jens Wildhagen, Hannover (DE)

(21) Appl. No.: 18/934,435

(22) Filed: Nov. 1, 2024

(30) Foreign Application Priority Data
Nov. 3, 2023 (DE) 10 2023 130 417.8

(51) Int. Cl. A61N 1/36; A61N 1/05

(52) U.S. Cl. CPC A61N 1/36178; A61N 1/0551; A61N 1/36071

(57) ABSTRACT

A medical arrangement for electrical neurostimulation includes an insertion aid with an insertion shaft which has a proximal end, a distal end and a lumen longitudinally extended between the proximal end and the distal end, a stimulator with a stimulator shaft which has a distal stimulator end with a stimulation electrode designed to deliver electric current pulses, and which has an electrical conductor, the distal end of which forms the stimulation electrode or is connected to the stimulation electrode. The arrangement is transferable between first and second configurations. In the first configuration, the stimulator shaft is inserted into the lumen via the proximal end of the insertion shaft and the distal stimulator end protrudes beyond the distal end. In the second configuration, the stimulator shaft is proximally pulled out of the lumen of the insertion shaft and the insertion aid and the stimulator are separated from one another.

### CROSS-REFERENCE TO RELATED APPLICATION(S)

This application claims priority under 35 U.S.C. § 119 to German Application No. 10 2023 130 417.8, filed on Nov. 3, 2023, the content of which is incorporated by reference herein in its entirety.

### FIELD

The present disclosure relates to a medical arrangement for electrical neurostimulation.

### BACKGROUND

Electrical neurostimulation (in short: neurostimulation or neuromodulation) is an established form of therapy for treating acute or chronic pain, and provides for the delivery of electric current pulses in the immediate vicinity of a nerve, in order to influence the conductive behaviour thereof for pain impulses.

### SUMMARY

It is an object of the present disclosure to provide a medical arrangement for electrical neurostimulation which is of simple construction and can be used in a variety of ways.

This object is achieved by providing a medical arrangement with the features of claim 1. Advantageous embodiments are specified in the dependent claims.

The medical arrangement according to the present disclosure is configured for electrical neurostimulation and comprises an insertion aid and a stimulator. The insertion aid comprises a longitudinally extended insertion shaft with a proximal shaft end, a distal shaft end and a lumen longitudinally extended between the proximal shaft end and the distal shaft end. The stimulator comprises a longitudinally extended stimulator shaft with a distal stimulator end and an electrical conductor. The distal stimulator end comprises at least one stimulation electrode which is configured to deliver electric current pulses. A distal end of the electrical conductor is connected to the at least one stimulation electrode or forms the at least one stimulation electrode. The medical arrangement according to the present disclosure is transferable between a first configuration and a second configuration. In the first configuration, the stimulator shaft is inserted into the lumen via the proximal shaft end of the insertion shaft and the distal stimulator end protrudes beyond the distal shaft end. In the second configuration, the stimulator shaft is proximally pulled out of the lumen of the insertion shaft and the insertion aid and the stimulator are separated from one another. The first configuration can be used for neurostimulation by means of said delivery of the electric current pulses via the at least one stimulation electrode of the stimulator. In the second configuration, a local anaesthetic can be delivered via the lumen of the insertion aid. The insertion aid consequently has an advantageous multiple function and on one hand is used for a simplified insertion of the stimulator and on the other hand is used as a type of capillary or catheter for delivering the local anaesthetic. As a result of the functional separation between neurostimulation on one hand and delivery of the local anaesthetic on the other hand, the stimulator can be constructed in the simplest possible manner. The stimulator preferably does not have a lumen for delivering a local anaesthetic. The lumen of the insertion aid can be designed, as an alternative or in addition to said delivery of the local anaesthetic, to accommodate a catheter which is designed to deliver a/the local anaesthetic.

In one embodiment, the stimulator shaft consists of the electrical conductor, wherein the stimulator shaft has no electrical insulation, and wherein, in the first configuration, the insertion shaft forms an electrically insulating sheath for the electrical conductor. This embodiment allows for a further simplified design of the stimulator. The simplified design is achieved by the fact that the stimulator shaft consists of the electrical conductor. The distal end of the electrical conductor forms the at least one stimulation electrode. The stimulator shaft/electrical conductor itself has no electrical insulation. The insulation function is taken over by the insertion shaft, which, in the first configuration of the medical arrangement, forms an electrically insulating sheath for the electrical conductor, as it were.

In one embodiment, the stimulator shaft has an electrically insulating sheath which encases the electrical conductor in portions, wherein the distal stimulator end is not encased by the electrical conductor, and wherein the stimulator end that protrudes beyond the distal shaft end of the insertion shaft in the first configuration has a first end portion which is not encased by the sheath and has the at least one stimulation electrode, and has a second end portion which is encased by the sheath. By dividing the stimulator end into the first end portion and the second end portion, precisely positioned delivery of the electric current pulses can take place. The delivery takes place via the at least one stimulation electrode which is arranged or formed on the first end portion. For this purpose, the first end portion is not encased by the electrically insulating sheath. The second end portion, by contrast, is electrically insulated. In the first configuration, both end portions protrude distally beyond the distal shaft end of the insertion shaft.

In one embodiment, the distal stimulator end has a plurality of stimulation electrodes which are configured to separately deliver electric current pulses. The plurality of stimulation electrodes for the separate delivery of electric current pulses allow the neurostimulation to be optimized. In one embodiment, the plurality of stimulation electrodes are arranged or formed proximodistally and/or spaced apart from one another along a longitudinal axis of the stimulator shaft on the distal stimulator end. In one embodiment, the plurality of stimulation electrodes are alternatively or additionally arranged or formed spaced apart from one another in the circumferential direction of the distal stimulator end. In one embodiment, at least one of the plurality of stimulation electrodes forms a counter electrode for the remaining stimulation electrodes.

In one embodiment, the medical arrangement further comprises at least one counter electrode, the at least one counter electrode being designed as a skin electrode for application to the skin of a patient or the at least one counter electrode is arranged on the stimulator end. The at least one counter electrode is used as a counter pole for the at least one stimulation electrode. For delivery of the electric current pulses, a voltage difference is produced between the at least one stimulation electrode and the counter electrode. In one embodiment, the at least one counter electrode is a skin electrode for application to the skin. Such skin electrodes are known to a person skilled in the art and are commercially available in various forms. By using such a skin electrode as a counter electrode, the construction of the medical arrangement can be further simplified. In one embodiment, the at least one counter electrode is arranged on the stimulator end. Provided that the stimulator end has a plurality of electrodes, one of the plurality of electrodes can function as a counter electrode.

In one embodiment, the stimulator shaft has a lumen longitudinally extended between the distal stimulator end and a proximal stimulator end, which lumen is configured to deliver a local anaesthetic. As a result, the stimulator can be used on one hand for neuromodulation and on the other hand for delivery of the local anaesthetic. While this allows the medical arrangement to be used in a variety of ways, the construction of the stimulator is less simple when compared to embodiments without lumen and is therefore less advantageous.

In one embodiment, the medical arrangement further comprises a control device being connected or connectable to the electrical conductor of the stimulator shaft, wherein the control device is configured to control the delivery of the electric current pulses via the at least one stimulation electrode. In one embodiment, the control device is an integral part of the stimulator. In one embodiment, the control device is a separate unit and electrically connectable or connected to the stimulator.

In one embodiment, the control device is connected or connectable to the plurality of stimulation electrodes, wherein the control device is configured to control the delivery of the separate electric current pulses. In this embodiment, the control device is designed to separately activate the plurality of stimulation electrodes. This allows the pain-reducing effect of the neuromodulation to be optimized. The control device is connected or connectable to the plurality of stimulation electrodes via the electrical conductor of the stimulator shaft. It is understood that the stimulator shaft can also have a plurality of electrical conductors, wherein in each case one of the plurality of electrical conductors is assigned to one of the plurality of stimulation electrodes.

In one embodiment, the medical arrangement further comprises a catheter configured to deliver a local anaesthetic, wherein the medical arrangement is transferable into a third configuration in which the catheter, instead of the stimulator shaft, is inserted into the lumen of the insertion aid. The catheter can also be referred to as a pain catheter and has a construction known to a person skilled in the art. By adding said catheter to the medical arrangement, a more varied range of uses can be achieved. In particular, there is no need for a specific configuration of the insertion aid which allows for delivery of the local anaesthetic via the lumen of the insertion shaft.

### BRIEF DESCRIPTION OF THE DRAWINGS

Further advantages and features of the present disclosure emerge from the claims and from the following description of preferred exemplary embodiments of the present disclosure, which are presented with reference to the drawings. In the drawings:

FIG. 1 shows a schematically simplified illustration of an embodiment of a medical arrangement for electrical neurostimulation with an insertion aid illustrated in longitudinal section and a stimulator, wherein the insertion aid and the stimulator are illustrated proximally cut off;

FIG. 2 shows the medical arrangement according to FIG. 1, wherein the stimulator is pushed distally into the insertion aid;

FIG. 3 shows, in a schematic block illustration, a control device of the medical arrangement according to FIGS. 1 and 2;

FIG. 4 shows a further embodiment of a medical arrangement according to the present disclosure in a view corresponding to FIG. 2;

FIG. 5 shows a further embodiment of a medical arrangement according to the present disclosure in a view corresponding to FIG. 2;

FIG. 6 shows a further embodiment of a medical arrangement according to the present disclosure in a view corresponding to FIG. 2;

FIG. 7 shows a further embodiment of a medical arrangement according to the present disclosure in a view corresponding to FIG. 2;

FIG. 8 shows, in a schematic block illustration, a counter electrode of the medical arrangements according to FIGS. 1 to 7;

FIG. 9 shows a further embodiment of a stimulator according to the present disclosure with a lumen which is illustrated partially cut free; and

FIG. 10 shows the insertion aid of the medical arrangement according to FIGS. 1 and 2 together with a catheter which, instead of the stimulator, is pushed into the insertion aid.

### DETAILED DESCRIPTION

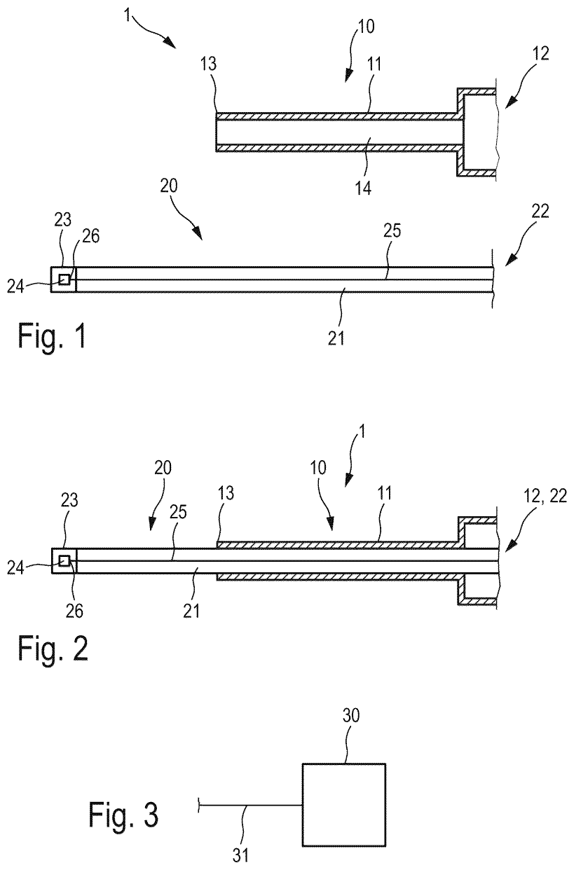

According to FIGS. 1 and 2, a medical arrangement 1 is provided for electrical neurostimulation in pain therapy and comprises an insertion aid 10 and a stimulator 20.

The insertion aid 10 comprises a longitudinally extended insertion shaft 11 with a proximal shaft end 12 and a distal shaft end 13. For simplified illustration, the insertion shaft 11 is cut off in the region of the proximal shaft end 12. The insertion shaft 11 has a longitudinally extended lumen 14 between the proximal shaft end 12 and the distal shaft end 13. The insertion aid 10 can also be referred to as a capillary.

The stimulator 20 comprises a longitudinally extended stimulator shaft 21 with a proximal stimulator end 22 and a distal stimulator end 23. For the purposes of simplified illustration, the stimulator shaft 21 is cut off in the region of the proximal stimulator end 22. The distal stimulator end 23 has at least one stimulation electrode 24 which is configured to deliver electric current pulses. Furthermore, the stimulator shaft 21 comprises an electrical conductor 25. The distal end 26 thereof is connected to the at least one stimulation electrode 24 here. Alternatively, the distal end can form the stimulation electrode.

In FIG. 2, the medical arrangement 1 is shown in a first configuration in which the stimulator shaft 21 is inserted into the lumen 14 via the proximal shaft end 12 of the insertion shaft 11 and the distal stimulator end 23 protrudes beyond the distal shaft end 13.

In FIG. 1, the medical arrangement 1 is shown in a second configuration in which the stimulator shaft 21 is proximally pulled out of the lumen 14 of the insertion shaft 11 and the insertion aid 10 and the stimulator are separated from one another.

The first configuration (FIG. 2) serves for neuromodulation by means of said delivery of electric current pulses via the at least one stimulation electrode 24. For this purpose, the distal stimulator end 23 is positioned, along with the at least one stimulation electrode 24, in the immediate vicinity of a nerve to be anaesthetized. The conductive behaviour of the nerve for pain impulses is influenced by the electric current pulses emitted from the stimulation electrode 24. This therapeutic approach to the treatment of acute or chronic pain is known to a person skilled in the art.

In the second configuration (FIG. 1), the insertion aid 10 serves to administer a local anaesthetic. For this purpose, the local anaesthetic can be introduced into the lumen 14 via the proximal shaft end 12 and delivered in a locally restricted manner via the distal shaft end 13 to the nerve to be anaesthetized. The proximal shaft end 12 can be provided with a fluid connector, an injection port or the like.

For controlling of the at least one stimulation electrode 24, the medical arrangement 1 further comprises a control device 30. The control device 30 is shown as a function block in a schematically simplified manner in FIG. 3 and is connected or connectable to the electrical conductor 25 via a control line 31. The control device 30 is configured to control the delivery of the electric current pulses via the at least one stimulation electrode 24.

In the embodiment shown in FIGS. 1 to 3, the control device 30 is presented locally separated from the stimulator 20 and forms a separate unit. In an embodiment that is not shown in the figures, the control device is integrated into the stimulator.

In the other figures, further embodiments of medical arrangements 1a, 1b, 1c, 1d are shown. The further medical arrangements 1a to 1d are substantially identical to the medical arrangement 1 in terms of their structure and function. Therefore, to avoid repetition, principally the main differences between the arrangements 1a to 1d and the arrangement 1 are explained hereinafter. Moreover, and unless described otherwise, the disclosure relating to the medical arrangement 1 also applies mutatis mutandis to the further medical arrangements 1a to 1d. Functionally identical components and/or portions are denoted by identical reference numbers with the addition of lower-case letters.

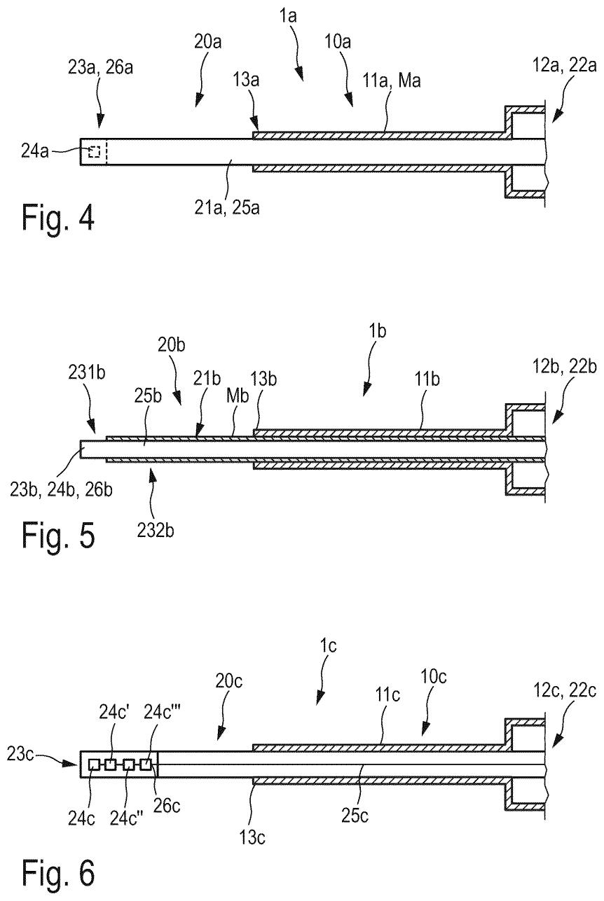

In the medical arrangement 1a according to FIG. 4, the stimulator shaft 21a consists of the electrical conductor 25a. The stimulator shaft 21a/the electrical conductor 25a itself has no electrical insulation. Instead, in the first configuration as shown in FIG. 4, the insertion shaft 11a forms an electrically insulating sheath Ma for the electrical conductor 25a. The distal end 26a of the electrical conductor 25a forms the distal stimulator end 23a. In addition, the electrical conductor 25a simultaneously forms at least one stimulation electrode 24a in the region of its distal end 26a. The distal stimulator end 23a and the stimulation electrode 24a are indicated by dashed lines in FIG. 4, wherein the shape and dimension shown are to be understood purely as examples. In principle, the entire proportion of the electrical conductor 25a that protrudes beyond the distal shaft end 13a forms a/the stimulation electrode.

In the medical arrangement 1b according to FIG. 5, the stimulator shaft 21b has an electrically insulating sheath Mb which encases the electrical conductor 25b at least in portions. In the region of the distal stimulator end 23b, the electrical conductor 25b lies free. There is no sheath present there. In the first configuration of the medical arrangement 1b, as shown in FIG. 5, the stimulator end 23b that protrudes beyond the distal shaft end 13b has a first (non-insulated) end portion 231b and a second (electrically insulated) end portion 232b. The first end portion 231b forms the at least one stimulation electrode 24b.

In the medical arrangement 1c according to FIG. 6, the distal stimulator end 23c has a plurality of stimulation electrodes 24c, 24c′, 24c″, 24c′″. These are configured to separately deliver electric current pulses. By separately delivering the electric current pulses via the plurality of stimulation electrodes 24c, 24c′, 24c″, 24c″, locally targeted delivery of the electric current pulses, and thus optimization of the neurostimulation in terms of the attainable reduction in pain, can be achieved. In the embodiment shown in FIG. 6, the plurality of stimulation electrodes 24c, 24c′, 24c″, 24c′″ are arranged proximodistally spaced apart from one another on the distal stimulator end 23c.

Alternatively or additionally, the plurality of stimulation electrodes 24c, 24c′, 24c″, 24c′″ can be spaced apart from one another in the circumferential direction of the distal stimulator end 23c. The electrical contacting of the plurality of stimulation electrodes 24c, 24c′, 24c″, 24c′″ takes place by means of the electrical conductor 25c. It is understood that the stimulator shaft 21c can have a plurality of electrical conductors, wherein in each case one of the electrical conductors is used to contact and/or form one of the stimulation electrodes.

It is furthermore understood that the control device 30 according to FIG. 3 can also be present in the medical arrangements la, 1b, 1c, 1d. Provided that a plurality of stimulation electrodes are present, the control device 30 is preferably configured to control the delivery of the separate electric current pulses via the plurality of stimulation electrodes.

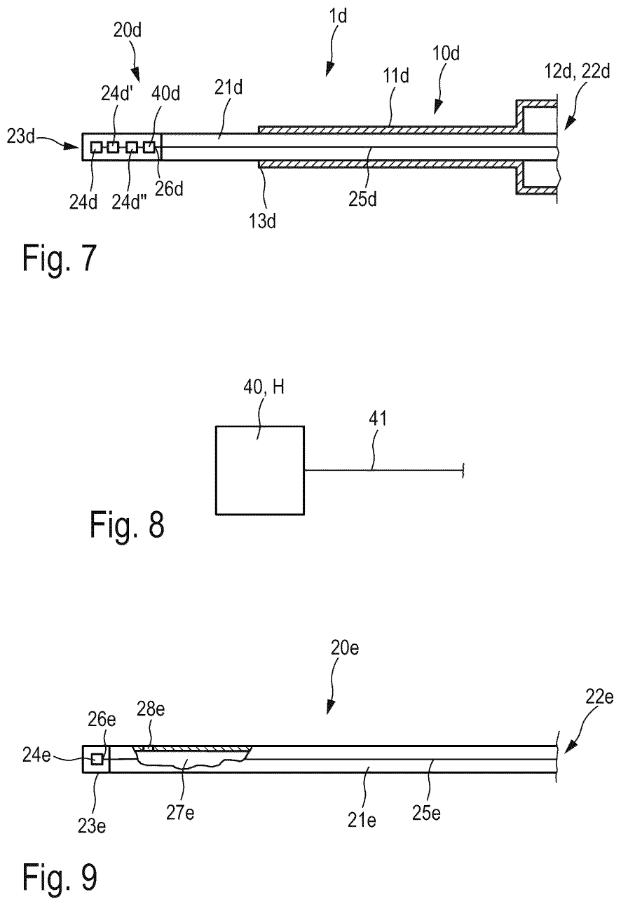

In the medical arrangement 1d according to FIG. 7, a counter electrode 40d is present and arranged on the distal stimulator end 23d. The counter electrode 40d can be electrically contacted via a separate electrical conductor or via a separate wire harness of the electrical conductor 25d. The counter electrode 40d and the plurality of stimulation electrodes 24d, 24d′, 24d″ have different electric polarities.

FIG. 8 shows a counter electrode 40 in the form of a skin electrode H. The skin electrode H is configured for application to the skin of a patient and is connected or connectable to the control device 30 via a signal line 41. The counter electrode 40 can be part of the medical arrangements 1, 1a, 1b, 1c.

FIG. 9 shows a stimulator 20e which, in contrast to the stimulators 20, 20a, 20b, 20c, 20d, has a lumen 27e with at least one outlet opening 28e. The lumen 27e is longitudinally extended between the proximal stimulator end 22e and the distal stimulator end 23e and opens at one end into the outlet opening 28e. The lumen 27e is configured to deliver a local anaesthetic through the outlet opening 28e.

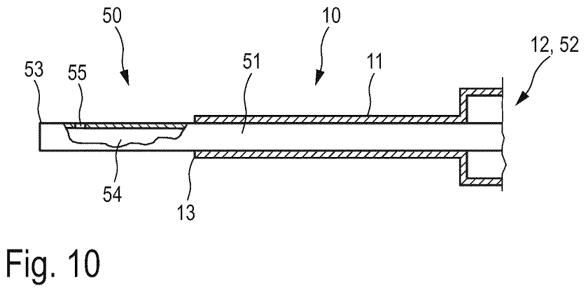

FIG. 10 shows the medical arrangement according to FIGS. 1 and 2 in a third configuration. In the third configuration, instead of the stimulator 20, a catheter 50 is pushed into the lumen 14 of the insertion shaft 11. The catheter 50 has a catheter shaft 51 with a proximal catheter end 52 and a distal catheter end 53. Furthermore, the catheter shaft 51 has a lumen 54 longitudinally extended between the proximal catheter end 52 and the distal catheter end 53, which lumen 54 has at least one outlet opening 55. The catheter 50 can also be referred to as a pain catheter and serves to deliver a local anaesthetic via the lumen 54 through the outlet opening 55.

It is understood that individual features of the embodiments explained above can be combined with one another to form further embodiments.

1. A medical arrangement for electrical neurostimulation, the medical arrangement comprising:
an insertion aid with an insertion shaft that is longitudinally extended and has a proximal shaft end, a distal shaft end and a lumen longitudinally extended between the proximal shaft end and the distal shaft end; and
a stimulator with a stimulator shaft that is longitudinally extended and has a distal stimulator end with at least one stimulation electrode which is configured to deliver electric current pulses, and which has an electrical conductor, a distal end of the electrical conductor forming the at least one stimulation electrode or is connected to the at least one stimulation electrode;
wherein the medical arrangement is transferable between a first configuration and a second configuration,
wherein, in the first configuration, the stimulator shaft is inserted into the lumen via the proximal shaft end of the insertion shaft and the distal stimulator end protrudes beyond the distal shaft end, and
wherein, in the second configuration, the stimulator shaft is proximally pulled out of the lumen of the insertion shaft and the insertion aid and the stimulator are separated from one another.

2. The medical arrangement according to claim 1, wherein the stimulator shaft comprises the electrical conductor, wherein the stimulator shaft has no electrical insulation, and wherein, in the first configuration, the insertion shaft forms an electrically insulating sheath for the electrical conductor.

3. The medical arrangement according to claim 1, wherein the stimulator shaft has a sheath that is electrically insulating sheath and encases the electrical conductor in portions, wherein the distal stimulator end that protrudes beyond the distal shaft end in the first configuration has a first end portion which is not encased by the sheath and has the at least one stimulation electrode, and has a second end portion which is encased by the sheath.

4. The medical arrangement according to claim 1, wherein the distal stimulator end has a plurality of stimulation electrodes which are configured to separately deliver electric current pulses.

5. The medical arrangement according to claim 4, further comprising a control device being connected or connectable to the electrical conductor of the stimulator, wherein the control device is configured to control delivery of the electric current pulses via the at least one stimulation electrode.

6. The medical arrangement according to claim 5, wherein the control device is connected or connectable to the plurality of stimulation electrodes, wherein the control device is configured to control delivery of the electric current pulses.

7. The medical arrangement according to claim 1, further comprising at least one counter electrode, the at least one counter electrode being designed as a skin electrode for application to a patient's skin or the at least one counter electrode is arranged on the distal stimulator end.

8. The medical arrangement according to claim 1, wherein the stimulator shaft has a lumen longitudinally extended between the distal stimulator end and a proximal stimulator end, which lumen is configured to deliver a local anaesthetic.

9. The medical arrangement according to claim 1, further comprising a catheter configured to deliver a local anaesthetic, wherein the medical arrangement is transferable into a third configuration in which the catheter, instead of the stimulator shaft, is inserted into the lumen of the insertion aid.
